(19) United States
(12) Patent Application Publication
(10) Pub. No.: US 2016/0095897 A1
(43) Pub. Date: Apr. 7, 2016

(54) PAR-4 ANTAGONISTS FOR USE IN THE TREATMENT OR PREVENTION OF INFLUENZA VIRUS TYPE A INFECTIONS

(71) Applicants: INSTITUT NATIONAL DE LA RECHERCHE AGRONOMIQUE, Paris (FR); HOSPICES CIVILS DE LYON, Lyon (FR); UNIVERSITE CLAUDE BERNARD LYON 1, Villeurbanne (FR)

(72) Inventors: Beatrice RITEAU, Craponne (FR); Ba Vuong LE, Lyon (FR); Bruno LINA, Lyon (FR)

(21) Appl. No.: 14/785,879

(22) PCT Filed: Apr. 22, 2014
(86) PCT No.: PCT/EP2014/058052

§ 371 Date: Oct. 21, 2015

(30) Foreign Application Priority Data
Apr. 22, 2013 (FR) 13305524.4

(51) Int. Cl. A61K 38/16; A61K 45/06

(52) U.S. Cl. CPC A61K 38/16; A61K 45/06

(57) ABSTRACT

The present invention provides methods and compositions (such as pharmaceutical compositions) comprising PAR4 antagonists for treating or preventing influenza virus type A infections, in particular H1N1 infection. PAR4 antagonists may be combined with a PAR2 agonist or a PAR1 antagonist.

### FIELD OF THE INVENTION

The present invention provides methods and compositions (such as pharmaceutical compositions) for treating or preventing influenza virus type A infections.

### BACKGROUND OF THE INVENTION

Epidemic viral infections are responsible for significant worldwide loss of life and income in human illnesses ranging from the common cold to life-threatening influenza, West Nile and HIV infections. Timely detection, diagnosis and treatment are key in limiting spread of disease in epidemic, pandemic and epizootic settings. In particular, prophylactic and therapeutic agents that rapidly inhibit viral assembly and propagation are particularly useful in treatment regimens.

Influenza virus of type A (IAV) causes acute respiratory infections that are highly contagious and afflict humans and animals with significant morbidity and mortality. Thus, there is a need in the clinical arts for new and improved anti-viral medicinal agents. This invention meets these needs.

Activation of host innate immune system aims at controlling the spreading and deleterious effects of IAV infection. However, excessive inflammatory response, due to a dysregulation of cytokine release and strong recruitment of neutrophils at the site of infection, may also mediate severe lung inflammation and increased pathogenesis of IAV. Cytokine dysregulation during IAV infection is thus often associated with fatal outcome of IAV.

The sites of virus replication in the respiratory tract represent complex microenvironments, in which extracellular proteases are present in large amounts. Some of these proteases (trypsin, tryptase) can play a role both in virus replication (Riteau B. et al. 2006; LeBouder F. et al. 2008) and innate immune responses as they are important mediators of inflammatory processes through the activation of a family of receptors called Protease-Activated Receptors (PARs) (Steinhoff M. et al. 2005; Vergnolle N. et al. 2008).

To date four PARs, activated by different proteases, have been cloned (PAR1-4). After cleavage of the receptor by proteases, the newly released amino-terminal sequence binds and activates internally the receptor.

The role of PAR4 in lung IAV infection has never been documented or in virus infection in general. However elevated PAR4 levels have been observed in the airways of IAV-infected mice (Lan R S. et al. 2004), suggesting a role for this receptor in the pathogenesis of viral disease. The specific role for PAR4 activation/inactivation in vivo or in vitro has never been addressed in the context of virus infection.

### SUMMARY OF THE INVENTION

The invention relates to a PAR4 antagonist for use in the treatment or prevention of an influenza virus type A infection.

### DETAILED DESCRIPTION OF THE INVENTION

The present invention provides methods and compositions (such as pharmaceutical compositions) for treating or preventing influenza virus type A infections.

The inventors indeed investigated the role of PAR4 in influenza pathogenesis and have shown that activation of PAR4 led to increased pathogenicity of IAV infection whereas PAR4 antagonist protects against IAV infection.

Accordingly, a first aspect of the invention relates to a PAR4 antagonist for use in the treatment or prevention of an influenza virus type A infection.

As used herein, the term “influenza virus type A infection” refers to any infection caused by an influenza virus type A without consideration of serotype based on hemagglutinine (H1 to H15) and neuraminidase (N1 to N9) expression. Exemplary influenza virus type A that are contemplated by the invention include but are not limited to H1N1, H2N2, H3N2, H5N1, H7N7, H1N2, H9N2, H7N2, H7N3, and H10N7. In a preferred embodiment, influenza virus type according to the present invention is H1N1.

In its broadest meaning, the term “treating” or “treatment” refers to reversing, alleviating, inhibiting the progress of influenza virus type A infection, preferably inhibiting the influenza virus type A proliferation. In particular, “prevention” or “prophylactic treatment” of influenza virus type A infections may refer to the administration of the compounds of the present invention that prevent the symptoms of influenza virus type A infections.

The dosage and frequency of administration can vary depending on whether the treatment is prophylactic or therapeutic. In prophylactic applications, a relatively low dosage is administered at relatively infrequent intervals over a long period of time. Some subjects continue to receive treatment for the rest of their lives. In therapeutic applications, a relatively high dosage at relatively short intervals is sometimes required until progression of the disease is reduced or terminated, and preferably until the subject shows partial or complete amelioration of symptoms of disease. Thereafter, the subject can be administered a prophylactic regime.

In prophylactic applications, compositions containing the antagonist of PAR4 are administered to a patient not already suffering from influenza virus type A infection. Rather, they are directed to a subject who is at the risk of, or has a predisposition, to developing such a disorder. Such applications allow the subject to enhance the patient's resistance or to retard the progression of a from influenza virus type A infection.

As used herein, the term “protease activated receptor-4”, “proteinase activated receptor-4” or “PAR4” or “PAR-4” interchangeably refer to a G-protein-coupled receptor that is activated by thrombin cleavage thereby exposing an N-terminal tethered ligand.

The term may include naturally occurring PAR4 and variants and modified forms thereof.

The PAR4 can be from any source, but typically is a mammalian (e.g., human and non-human primate) PAR4, particularly a human PAR4.

The amino sequence of human proteinase-activated receptor 4 precursor is published as GenBank accession number NP_{—}003941.

The term “antagonist,” as used herein, refers to an agent that is capable of specifically binding and inhibiting signaling through a receptor to fully block or detectably inhibit a response mediated by the receptor.

For example, as used herein the term “PAR4 antagonist” is a natural or synthetic compound which binds and inactivates fully or partially PAR4 for initiating a pathway signalling and further biological processes. PAR-4 antagonistic activity may assessed by various known methods which fall within the general knowledge of the person skilled in the art.

In one embodiment, a PAR4 antagonist according to the invention may be a peptide, a peptide mimetic, a small molecule organic compound, an aptamer, a pepducin, a polynucleotide or an antibody.

Preferably, said PAR-4 antagonist is a pepducin.

Pepducins are modified peptides comprising a first domain of a first intracellular loop (il loop) or a fragment thereof of a G protein coupled receptor (GPCR) and a second domain, attached to the first domain said second domain being a naturally or non-naturally occurring cell-penetrating or membrane-tethering moiety. In a specific embodiment, pepducin can be synthesized as retro-inverso isomers, which include peptides of reverse sequence and chirality. See, e.g., Jameson et al. Nature 368:744-746 (1994) and Brady et al. Nature 368:692-693 (1994). The net result of combining D-enantiomers and reverse synthesis is that the positions of carbonyl and amino groups in each amide bond are exchanged, while the position of the side-chain groups at each alpha carbon is preserved. For example, if the peptide model is a peptide formed of L-amino acids having the sequence ABC, the retro-inverso peptide analog formed of D-amino acids would have the sequence CBA. The procedures for synthesizing a chain of D-amino acids to form the retro-inverso peptides are known in the art.

Also useful are amino-terminal blocking groups such as t-butyloxycarbonyl, acetyl, theyl, succinyl, methoxysuccinyl, suberyl, adipyl, azelayl, dansyl, benzyloxycarbonyl, fluorenylmethoxycarbonyl, methoxyazelayl, methoxyadipyl, methoxysuberyl, and 2,4,-dinitrophenyl. Blocking the charged amino- and carboxy-termini of the peptides would have the additional benefit of enhancing passage of the peptide through the hydrophobic cellular membrane and into the cell.

“Cell-penetrating moieties” include compounds or functional groups which mediate transfer of a substance from an extracellular space to an intracellular compartment of a cell. Cell-penetrating moieties shuttle a linked substance {e.g., a GPCR peptide or fragment of the invention) into the cytoplasm or to the cytoplasmic space of the cell membrane. For example, a cell penetrating moiety is a hydrophobic moiety. The hydrophobic moiety is, e.g., a mixed sequence peptide or a homopolymer peptide such as polyleucine or polyarginine at least about 11 amino acids long. The substance may be a peptide such as a GPCR fragment or peptidomimetic of the invention. The cell penetrating moiety may include at least 10 contiguous amino acids, e.g., 1-15 amino acids of a GPCR transmembrane helix domain.

“Membrane-tethering moieties” include compounds or functional groups which associate with or bind to a cell membrane. Thus, the membrane-tethering moiety brings the substance to which the membrane-tethering moiety is attached (i.e., the GPCR fragment or peptidomimetic of the invention) in close proximity to the membrane of a target cell.

The cell membrane is eukaryotic or prokaryotic. The membrane-tethering moiety is desirably a hydrophobic moiety. The hydrophobic moiety can include a mixed sequence peptide or a homopolymer peptide such as polyleucine or polyarginine less than 10 amino acids long. The membrane-tethering moiety can include at least one to seven contiguous amino acids of a GPCR transmembrane helix domain. Preferably, the membrane-tethering moiety is at least 10 contiguous amino acids (but less than 16 amino acids) of a GPCR transmembrane domain; more preferably, the membrane-tethering moiety is at least 15 contiguous amino acids of a GPCR transmembrane domain. Membrane-tethering moieties also include cholesterol, phospholipids, steroids, sphingosine, ceramide, octyl-glycine, 2-cyclohexylalanine, or benzolylphenylalanine. Other membrane-tethering moieties include C1 or C2 acyl groups, or a C3-C8 fatty acid moiety such as propionoyl (C3); butanoyl (C4); pentanoyl (C5); caproyl (C6); heptanoyl (C7); and capryloyl (C8). The membrane-tethering moiety may be attached to the C-terminal amino acid, the N-terminal amino acid, or to an amino acid between the N-terminal and C-terminal amino acid of the GPCR fragment in the pepducin.

The pepducin approach is highly advantageous since it allows the rich diversity of intracellular receptor structures to be exploited both for generation of new therapeutic agents and for delineation of the mechanisms of receptor-G protein coupling under in vivo conditions. The pepducins implemented by this strategy may also prove to be more selective to the extent that the pepducins primarily target the receptor rather than the G protein. Pepducin antagonists can be generated which are tailored to these receptors, and may be useful in determining which signaling pathways are activated by a given receptor in the context of its native environment.

Pepducins may inhibit thrombin-induced platelet activation by impairing the function of the PAR-4. However, the inventors met the burden to identify, for the very first time, a new role of pepducin targeting PAR4 in the treatment of IAV infection.

Preferably, said PAR4 antagonist is pepducin P4 pal-10.

As used herein, the term “pepducin P4pal-10”, also called “N-palmitoyl-SGRRYGHALR-NH2” or “P4pal-10”, refers to a selective antagonist peptide of PAR4 having the following formula: _{C}65H111N21014 Said antagonist is described in the literature and is commercially available. Indeed, pepducin P4pal-10 is commercialised by the companies Incelligen and Polypeptide Laboratories.

In another embodiment, said PAR4 antagonist is tcY-NH2 (trans-cinnamoyl-YPGKF-NH2).

“tcY-NH2” (trans-cinnamoyl-YPGKF-NH2) is a peptide commercialized by Tocris Biosciences and Sigma Aldrich.

In another embodiment of the invention, a PAR4 antagonist is a small molecule organic compound. The term “small organic molecule” refers to a molecule of a size comparable to those organic molecules generally used in pharmaceuticals. The term excludes biological macromolecules (e. g., proteins, nucleic acids, etc.). Preferred small organic molecules range in size up to about 5000 Da, more preferably up to 2000 Da, and most preferably up to about 1000 Da.

In another embodiment of the invention, a PAR4 antagonist is an antagonist PAR4 antibodies or antigen-binding molecule. As used herein, unless otherwise defined, the term “antibody” includes both polyclonal and monoclonal antibodies, as well as antibody fragments having specific binding affinity for their antigen, including, but not limited to, Fv fragments, Fab fragments, Fab′ fragments, F(ab)′2 fragments, and single chain (sFv) engineered antibody molecules. The term further includes, unless specifically excluded, chimeric and humanized antibodies, as well as human antibodies in circumstances where such antibodies can be produced.

These anti-PAR4 agents are capable of antagonizing PAR4 mediated signaling activities, e.g., PAR4 mediated interleukin secretion. General methods for preparation of monoclonal or polyclonal antibodies are well known in the art. See, e.g., Harlow & Lane, Using Antibodies, A Laboratory Manual, Cold Spring Harbor Laboratory Press, Cold Spring Harbor, New York, 1998; Kohler & Milstein, Nature 256:495-497 (1975); Kozbor et al., Immunology Today 4:72 (1983); and Cole et al., pp. 77-96 in Monoclonal Antibodies and Cancer Therapy, 1985.

In addition, specific PAR4 antagonist antibodies have been disclosed in the art.

Antibodies can be of any mammalian or avian origin, including human, murine (mouse or rat), donkey, sheep, goat, rabbit, camel, horse, or chicken. In some alternatives, the antibodies can be bispecific. The antibodies can be modified by the covalent attachment of any type of molecule to the antibody. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, or other modifications known in the art. Monoclonal antibodies can be prepared using a wide variety of techniques known in the art including the use of hybridoma, recombinant, and phage display technologies, or a combination thereof.

For example, monoclonal antibodies can be produced using hybridoma techniques including those known in the art and taught, for example, in Harlow et al., “Antibodies: A Laboratory Manual”, (Cold Spring Harbor Laboratory Press, 2nd ed. 1988); Hammerling, et al., in: Monoclonal Antibodies and T-CeII Hybridomas 563-681 (Elsevier, N.Y., 1981), or by other standard methods known in the art.

The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. For example, suitable antibodies can be produced by phage display or other techniques.

Additionally, and not by way of limitation, human antibodies can be made by a variety of techniques, including phage display methods using antibody libraries derived from human immunoglobulin sequences and by the use of transgenic mice that are incapable of expressing functional endogenous immunoglobulins, but which can express human immunoglobulin genes. For example, the human heavy and light chain immunoglobulin gene complexes can be introduced randomly or by homologous recombination into mouse embryonic stem cells. The antibodies can also be produced by expression of polynucleotides encoding these antibodies.

Additionally, antibodies according to the present invention can be fused to marker sequences, such as a peptide tag to facilitate purification; a suitable tag is a hexahistidine tag. The antibodies can also be conjugated to a diagnostic or therapeutic agent by methods known in the art. Techniques for preparing such conjugates are well known in the art.

Other methods of preparing these monoclonal antibodies, as well as chimeric antibodies, humanized antibodies, and single-chain antibodies, are known in the art.

In addition to compounds which inhibit or suppress PAR4 biochemical or signaling activities, compounds which are capable of suppressing PAR4 expression or down-regulating PAR4 cellular levels can also be used in the practice of the present invention. Suppression of PAR4 expression or down-regulation of its cellular level refers to a decrease in or an absence of PAR4 expression in an examined cell (e.g., a cell which has been contacted with a PAR4 antagonist compound), as compared to PAR4 in a control cell (a cell not treated with the PAR4 antagonist compound). PAR4 level or expression can be decreased or reduced by at least about 10% (e.g., by 20%, 30%, 40%, 50%, 60%, 70%, 80%, or 90%), as compared to PAR4 level or expression in the control cell.

As indicated above, suppression of expression or down-regulation of PAR4 cellular levels can be carried out at either the level of transcription of the gene for PAR4 into mRNA or the translation of mRNA for PAR4 into the corresponding protein.

In some embodiments, inhibitory nucleotides are used to antagonize PAR4 mediated cardiac remodeling or other effects of PAR4 by suppressing PAR4 expression. These include short interfering RNA (siRNA), microRNA (miRNA), and synthetic hairpin RNA (shRNA), anti-sense nucleic acids, or complementary DNA (cDNA). In some preferred embodiments, a siRNA targeting PAR4 expression is used. Interference with the function and expression of endogenous genes by double-stranded RNA such as siRNA has been shown in various organisms. See, e.g., A. Fire et al., “Potent and Specific Genetic Interference by Double-Stranded RNA in Caenorhabditis elegans ” Nature 391 :806-811 (1998); J. R. Kennerdell & R. W. Carthew, “Use of dsDNA-Mediated Genetic Interference to Demonstrate that frizzled and frizzled 2 Act in the Wingless Pathway,” CeJ 95:1017-1026 (1998); F. Wianni & M. Zernicka- Goetz, “Specific Interference with Gene Function by Double-Stranded RNA in Early Mouse Development,” Nat. Cell Biol. 2:70-75 (2000). siRNAs can include hairpin loops comprising self-complementary sequences or double stranded sequences. siRNAs typically have fewer than 100 base pairs and can be, e.g., about 30 bps or shorter, and can be made by approaches known in the art, including the use of complementary DNA strands or synthetic approaches. Such double-stranded RNA can be synthesized by in vitro transcription of single-stranded RNA read from both directions of a template and in vitro annealing of sense and antisense RNA strands. Double-stranded RNA targeting PAR4 can also be synthesized from a cDNA vector construct in which a PAR4 gene (e.g., human PAR4 gene) is cloned in opposing orientations separated by an inverted repeat. Following cell transfection, the RNA is transcribed and the complementary strands reanneal. Double-stranded RNA targeting the PAR4 gene can be introduced into a cell (e.g., a tumor cell) by transfection of an appropriate construct.

Typically, RNA interference mediated by siRNA, miRNA, or shRNA is mediated at the level of translation; in other words, these interfering RNA molecules prevent translation of the corresponding mRNA molecules and lead to their degradation. It is also possible that RNA interference may also operate at the level of transcription, blocking transcription of the regions of the genome corresponding to these interfering RNA molecules.

The structure and function of these interfering RNA molecules are well known in the art and are described, for example, in R.F. Gesteland et al., eds, “The RNA World” (3^{rd} ed, Cold Spring Harbor Laboratory Press, Cold Spring Harbor, N.Y., 2006), pp. 535-565, incorporated herein by this reference. [0110] For these approaches, cloning into vectors and transfection methods are also well known in the art and are described, for example, in J. Sambrook & D. R. Russell, “Molecular Cloning: A Laboratory Manual” (3^{rd} ed., Cold Spring Harbor Laboratory Press, Cold Spring Harbor, 2001), incorporated herein by this reference.

In addition to double stranded RNAs, other nucleic acid agents targeting PAR4 can also be employed in the practice of the present invention, e.g., antisense nucleic acids. Antisense nucleic acids are DNA or RNA molecules that are complementary to at least a portion of a specific target mRNA molecule. In the cell, the single stranded antisense molecule hybridizes to that mRNA, forming a double stranded molecule. The cell does not translate an mRNA in this double-stranded form. Therefore, antisense nucleic acids interfere with the translation of mRNA into protein, and, thus, with the expression of a gene that is transcribed into that mRNA. Antisense methods have been used to inhibit the expression of many genes in vitro. See, e.g., C J. Marcus- Sekura, “Techniques for Using Antisense Oligodeoxy ribonucleotides to Study Gene Expression,” Anal. Biochem. 172:289-295 (1988); J. E. Hambor et al., “Use of an Epstein-Barr Virus Episomal Replicon for Anti-Sense RNA-Mediated Gene Inhibition in a Human Cytotoxic T-CeIl Clone,” Proc. Natl. Acad. Sci. U.S.A. 85:4010-4014 (1988); H Arima et al., “Specific inhibition of lnterleukin-10 Production in Murine Macrophage-Like Cells by Phosphorothioate Antisense Oligonucleotides,” Antisense Nucl. Acid Drug Dev. 8:319-327 (1998); and W.-F. Hou et al., “Effect of Antisense Oligodeoxynucleotides Directed to Individual Calmodulin Gene Transcripts on the Proliferation and Differentiation of PC12 Cells,” Antisense Nucl. Acid Drug Dev. 8:295-308 (1998), all incorporated herein by this reference. Antisense technology is described further in C. Lichtenstein & W. Nellen, eds., “Antisense Technology: A Practical Approach” (IRL Press, Oxford, 1997), incorporated herein by this reference. [0111] PAR4 polynucleotide sequences from human and many other mammals have all been delineated in the art. Based on the known sequences, inhibitory nucleotides (e.g., siRNA, miRNA, or shRNA) targeting PAR4 can be readily synthesized using methods well known in the art.

Other exemplary PAR4 antagonists that are contemplated by the invention include but are not limited to those described in the followings documents, which are incorporated by reference:
EP6667 345;
EP 1 166 785;
JP 2002080367;
EP 1 331 233;
US 2006106032;
WO 2009124103;
WO2008/086069;
WO2006/052723; and
Adams M N et al, Structure, function and pathophysiology of protease activated receptors, Pharmacol Ther. 2011 June; 130(3):248-82.

A further object of the invention relates a method for screening a PAR4 antagonist for use in the treatment or prevention of an influenza virus type A infection. For example, the screening method may measure the binding of a candidate compound to PAR4, or to cells or membranes bearing PAR4, or a fusion protein thereof by means of a label directly or indirectly associated with the candidate compound. Furthermore, the screening method may involve measuring or, qualitatively or quantitatively, detecting ability of said candidate compound to inactivate PAR4.

In a particular embodiment, the screening method of the invention comprises the steps consisting of:
a) providing a plurality of cells expressing PAR4 on their surface:
b) incubating said cells with a candidate compound;
c) determining whether said candidate compound binds to and inactivates PAR4; and
d) selecting the candidate compound that binds to and inactivates PAR4,

In a particular embodiment, the screening method of the invention may further comprising a step consisting of administering the candidate compound selected at step d) to an animal model of influenza virus type A infection to validate the protective effects of said candidate compound.

In general, such screening methods involve providing appropriate cells which express PAR4 on their surface. In particular, a nucleic acid encoding PAR4 may be employed to transfect cells to thereby express the receptor of the invention. Such a transfection step may be accomplished by methods well known in the art. In a particular embodiment, said cells may be selected from the group consisting of the mammal cells reported yet to express PAR4 (e.g. epithelial cells).

The screening method of the invention may be employed for determining a PAR4 antagonist by contacting such cells with compounds to be screened and determining whether such compound inactivates PAR4.

According to a one embodiment of the invention, the candidate compounds may be selected from a library of compounds previously synthesised, or a library of compounds for which the structure is determined in a database, or from a library of compounds that have been synthesised de novo or natural compounds. The candidate compound may be selected from the group of (a) proteins or peptides, (b) nucleic acids and (c) organic or chemical compounds (natural or not).

PAR4 inactivation with the candidate compound can be tested by various known methods of the man skilled in the art.

Another object of the invention relates to a method for treating or preventing a influenza virus type A infection comprising administering a subject in need thereof with an PAR4 antagonist.

As used herein, the term “subject” denotes a mammal, such as a pig and a primate. Preferably, a subject according to the invention is a human.

PAR4 antagonists may be administered in the form of a pharmaceutical composition, as defined below.

Preferably, the PAR4 antagonist of the invention is administered in a therapeutically effective amount. By a “therapeutically effective amount” is meant a sufficient amount the PAR4 antagonist according to the invention to treat or prevent influenza virus type A infections at a reasonable benefit/risk ratio applicable to any medical treatment.

It will be understood that the total daily usage of the compounds and compositions of the present invention will be decided by the attending physician within the scope of sound medical judgment. The specific therapeutically effective dose level for any particular patient will depend upon a variety of factors including the deficit being treated and the severity of the deficit; activity of the specific compound employed; the specific composition employed, the age, body weight, general health, sex and diet of the patient; the time of administration, route of administration, and rate of excretion of the specific compound employed; the duration of the treatment; drugs used in combination or coincidential with the specific polypeptide employed; and like factors well known in the medical arts. For example, it is well within the skill of the art to start doses of the compound at levels lower than those required to achieve the desired therapeutic effect and to gradually increase the dosage until the desired effect is achieved.

However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15,0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The PAR4 antagonist may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Preferably, the pharmaceutical composition according to the invention in preferably administered in an intranasal administration form.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action pf microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The PAR4 antagonist can be formulated into a composition in a neutral or salt form. Pharmaceutically acceptable salts include the acid addition salts (formed with the free amine groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils, The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The PAR4 antagonist may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

According to the present invention, the PAR4 antagonist may be formulated in combinations with one or more distinct active pharmaceutical agents, preferably active pharmaceutical agents for the treatment of influenza virus type A infection. Such agents may act by very different biochemical pathways to provide particularly beneficial therapeutic results and are well known by the person skilled in the art. According to the present invention the one or more active agents may be delivered as either co-administered monotherapy formulations, or as a single co-formulation.

In a preferred embodiment, one of the active agents is a PAR1 antagonist or a PAR2 agonist.

A further object of the invention relates to a pharmaceutical composition comprising:

(i) at least one PAR-4 antagonist, and

(ii) at least one Protease-Activated Receptors-2 (PAR-2) agonist or at least one PAR-1 antagonist.

A further object of the invention relates to products containing :

(i) at least one PAR-4 antagonist, and

(ii) at least one Protease-Activated Receptors-2 (PAR-2) agonist, or at least one PAR-1 antagonist as a combined preparation for simultaneous, separate, or sequential use for the treatment or prevention of an influenza virus type A infection in a subject.

As used herein, the term “PAR2” has its general meaning in the art and refers to Protease-Activated Receptor-2. The term may include naturally occurring PAR2 and variants and modified forms thereof. The PAR2 can be from any source, but typically is a mammalian (e.g., human and non-human primate) PAR2, particularly a human PAR2,

As used herein the term “PAR2 agonist” is a natural or synthetic compound which binds and activates PAR2 for initiating a pathway signalling and further biological processes. PAR-2 agonistic activity may assessed by various known methods. For example, the Hollenberg's method (Hollenberg, M. D., et al., Cati. J. Physiol. Pharmacol., 75,832-841 (1997)), the Kawabata's method (Kawabata, A., et al., J. Pharmacol. Exp. Ther., 288,358-370 (1999)) and the Hawthorne's method (Howthorne et al., A High-Throughput Microtiter Plate-Based Calcium Assay for the Study Of Protease-Activated Receptor 2 Activation, Analytical 13iochemistry 290,378-379 (2001)) may be used for assessing a PAR2 agonistic activity.

In one embodiment, a PAR2 agonist according to the invention may be a small organic molecule. Exemplary PAR2 agonists that are contemplated by the invention include but are not limited to those described in US Patent Application Publications Nos 2007123508 and 2008318960 that arc hereby incorporated by reference into the present disclosure. Other examples include those described in Graddil L R et al. 2008, and more particularly AC-55541 [N-[[1-(3-bromo-phenyl)-cth-(E)-ylidene-hydrazinocarbonyi}-(4-oxo-3,4-dihydro-phthalazin-1-yI)-methylj-benzamide] and AC-264613 [2-oxo-4-phenyipyrrolidine-3-carboxylic acid [t(3-bromo-phenyl)-(E/Z)-ethylidene}-hydrazide].

In another embodiment, a PAR2 agonist according to the invention is a PAR2 activating peptide that may be HOOC-SLIGRL-NH2 (SEQ ID NO: 5) or HOOC-SLIGKV-NH2 (SEQ ID NO: 6).

In another embodiment, a PAR2 agonist of the invention may be a PAR2 activating peptide derivative that may be selected from the group consisting of HOOC-LIGRLO-NH2, HOOC-Fluoryi-LIGRLO-NH2, and trans-cinnamoyl-LIGRLO (tc)-NH2.

Other PAR2 activating peptide derivatives that are contemplated by the invention include those described in International Patent Application Publication No W003/104268 (that is hereby incorporated by reference into the present disclosure) that are represented by the generai formula (I) or a salt thereof:

Z—(CH₂)—CO—NH-Leu-Ile-Gly-AA 1-AA2-CO—R (I)

wherein Z represents an aryl group which may or may not have a substituent or a heteroaryl group which may or may not have a substituent; n represents 0, 1 or 2 ; AA 1-AA2 represents Lys-Val or Arg-Leu; and R represents —OH or—NH2.

The aryl group as Z may be a carbon cyclic group of mono-ring type, multi-ring type or condensed ring type, with 6 to 30 carbon atoms, preferably 6 to 14 carbon atoms, specifically including for example phenyl group and naphtyl group, preferably. The heteroaryl group as Z may be a hetero-cyclic group of 5-te 7-membered mono-ring type, multi-ring type or condensed ring type, the group containing at least one to 3 nitrogen atoms, oxygen atoms or sulfur atoms within die ring and specifically including for example furyl group, thienyl group, pyridyl group or quinolyl group, preferably.

The aryl group or heteroaryl group as Z may or may not have a substituent, which includes but is not limited to any aryl group or heteroaryl group with no adverse effects on the activity of die inventive peptide derivative, specifically including for example a halogen atom, a lower alkyl group, a lower alkoxyl group, phenyl group, a phenyl-lower alkyl group, nitre group, amino group, hydroxyl group, and carboxyl group.

The halogen atom includes for example chlorine atom, fluorine atom, and bromine atom. The lower alkyl group is preferably n linear or branched lower alkyl group with one to 15 carbon atoms, preferably one to 6 carbon atoms, which includes for example methyl group and ethyl group. The lower alkoxyl group preferably includes a linear or branched lower alkoxyl group with one to 15 carbon atoms, preferably one to 6 carbon atoms, which includes for example methoxyl group and ethoxyl group.

The lower alkyl group in the phenyl-lower alkyl group includes alkylene groups including the lower alkyl group, for example methylene group and ethylene group.

Substituents for this lower alkyl group, tower alkoxyl group, phenyl group, and phenyl-lower alkyl group may additionally be substituted with a halogen atom and the like.

The group Z in the general formula (I) in accordance with the invention includes for example substituted or unsubstituted phenyl group, naphthyl group, furyl group, thienyl group, pyridyl group and quinolyl group, specifically including for example phenyl group, 4-methoxyphenyl group, 3-methoxyphenyl group, 2-methoxyphenyl group, 2,4-dimethoxyphenyi group, 3,5-dimethoxyphenyl group, 4-phenethylphcnyl group, 3-phenethylphenyl group, 2-phenethylphenyl group, 4-nitrophenyl group, 3-nitrophenyl group, 2-nitrophenyl group, 2,4-dinitrophenyl group, 3 ,4-dinitrophenyi group, 4-mcthylphenyl group, 3-methylphenyt group, 2-methyiphenyi group, 2,4-dimethylphenyl group, 3,5-dimethylphenyl group, 4-fluorophenyl group, 3-fluorophenyl group, 2-fluorophenyl group, 2,4-difluorophenyl group, 3,5-difluorophenyl group, 2,4, 5-trifluorophenyl group, 4-phenylphenyl group, 3-phenylphenyl group, 2-phcnylphcnyl group, 2.-furyt group, 3..furylgroup, 5-methoxy-2-furylgroup, 5-methyl-2-furylgroup, 1-naphthyl group, 2-naphthyl group, 4-methoxy-I-naphthyl group, 4-methyl- 1-naphthyl group, 4-methoxy-2-naphthyl group, 4-methyl-2-naphtyl group, 4-pyridyl group, 2-pyridyi group, 3-pyridyl group, 2-methyl-.4-pyridyl group, 4-methyl-2-pyridyl group, 2-thienyl group, 3-thienyl group, 3-methyl-2-thienyl group, 4-methyl-2-thienyl group, 4-methyl-3-thienyl group, 6-quinolyl group, 7-quinotyt group, 8-quinolyl group, 4-quinolyl group, 4-methyl-6-quinolyl group and the like.

In the general formula (I), in accordance with the invention, n represents 0, 1 or 2 and the group with the inferior letter “n” is bound to the group Z. When n is 0, the group Z in directly bound to carbonyl group; when n is t, the group z is bound through methylene group to carbonyl group; and when n is 2, the group Z is bound through ethylene group to carbonyl group.

R in the general formula (I) represents —OH or —NH₂, or the salt thereof.

In accordance with the invention, AA1-AA2 in the general formula (I) represents two types of amino acids bound together. The amino acid AA1, is preferably Lys or Arg, while AA2 in preferably Val or Leu. AA1 and AA2 are bound together in the sequence AAI-AA2 along the N-terminal to C-terminal direction. Preferably AA1-AA2 includes Lys-Val or Arg-Leu.

In another embodiment, a PAR2 agonist according to the invention is a protease that is known to activate PAR2. For example, trypsin and tryptase are the principal agonists of PAR2. Trypsin and tryptase cleave PAR2 to expose the tethered ligand SLIGRL (SEQ ID NO: 1) (rat and mouse PAR2), which then binds to conserved regions in extracellular loop II of the cleaved receptor. Certain coagulation factors can also activate PAR2 such as Factor VIIa or Factor Xa. Other examples include protease derived from epithelial cells such as maptriptase, human airway trypsin-like protease, and extra pancreatic tryptic enzymes.

In another embodiment the PAR2 agonist may consist in an antibody (the term including antibody fragment). In particular, the PAR2 agonist may consist in an antibody directed against the PAR2 in such a way that said antibody activates the receptor.

In another embodiment, the PAR2 agonist may be an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

Then after raising aptamers directed against PAR2 as above described, the skilled man in the art can easily select those activating PAR2.

As used herein, the term “protease activated receptor-1,” “proteinase activated receptor-1” or “PAR1” or “PAR-1” interchangeably refer to a G-protein-coupled receptor that is activated by thrombin cleavage thereby exposing an N-teiminal tethered ligand. PAR1 is also known as “thrombin receptor” and “coagulation factor Il receptor precursor.” See, for example, Vu, et al., Cell (1991) 64(6): 1057-68; Coughlin, et al, J Clin Invest (1992) 89(2):351-55; and GenBank Accession number NM_{—}001992. Intramolecular binding of the tethered ligand to the extracellular domain of PAR 1 elicits intracellular signaling and calcium flux. See, for example, Traynelis and Trejo, Curr Opin Hematol (2007) 14(3):230-5; and Hollenberg, et al, Can J Physiol Pharmacol. (1997) 75(7):832-41.

Typically, a PAR1 antagonist according to the invention may be a peptide, a peptide mimetic, a small molecule organic compound, an aptamer, a pepducin, a polynucleotide or an antibody.

In one embodiment, the administered PAR1 antagonist inhibits a PAR1 signaling activity. Some of these methods employ a PAR1 antagonist which is a peptidomimetic, e.g., RWJ-56110 or ([alpha]S)-W-[(1S)-3-amino-1-[[{phenylmethyl)amino]carbonyl] propyl]-[alpha]-[[[[[1-(2,6-dichlorophenyl)methyl]-3-(1-pyrrolidinylmethyl)-1H-indol-6-yi]amino]carbonyl]amino]-3,4-difluorobenzenepropanamide.

In another embodiment, a PAR1 antagonist is a small molecule organic compound. The term “small organic molecule” refers to a molecule of a size comparable to those organic molecules generally used in pharmaceuticals. The term excludes biological macromolecules (e. g., proteins, nucleic acids, etc.). Preferred small organic molecules range in size up to about 5000 Da, more preferably up to 2000 Da, and most preferably up to about 1000 Da. In a preferred embodiment, the PAR1 antagonist is the small organic molecule SCH-79797, which is (N3-cyclopropyl-7-{[4-(1-methylethyl)phenyl]methyl)-7H-pyrrolo[3,2-f]quinazoline-1,3-diamine).

In still another embodiment, the PAR1 antagonist may consist in an antibody (the term including antibody fragment). In particular, the PAR1 antagonist may consist in an antibody directed against the PAR1 in such a way that said antibody antagonise PAR1 mediated signaling activities, e.g. PAR1 mediated interleukin secretion. Specific PAR1 antagonist antibodies have been disclosed in the art. See, e.g., R. R. Vassallo, Jr. et al. “Structure-Function Relationships in the Activation of Platelet Thrombin Receptors by Receptor-Derived Peptides,” J. Biol. Chem. 267:6081-6085 (1992) (“Vassallo, Jr. et al. (1992”)); L. F. Brass et al., “Structure and Function of the Human Platelet Thrombin Receptor,” J. Biol. Chem. 267: 13795-13798 (1992) (“Brass et al. (1992)”); and R. Kaufmann et al., “Investigation of PAR-1-Type Thrombin Receptors in Rat Glioma C6 Cells with a Novel Monoclonal Anti-PAR-1 Antibody (Mab COR7-6H9), J. Neurocytol. 27:661-666 (1998) (“Kaufmann et al. (1998)”), both of which are incorporated herein by this reference.

Another aspect of the invention relates to a PAR4 antagonist for inhibiting replication of an influenza virus type A.

A further object of the invention relates to a method of testing whether a subject is predisposed to an influenza virus type A infection, which comprises the step of analyzing a biological sample from said subject for:

(i) detecting the presence of a mutation in the PAR4 gene and/or its associated promoter, and/or

(ii) analyzing the expression of the PAR4 gene,

As used herein, the term “biological sample” refers to any sample from a subject such as blood or serum.

Typical techniques for detecting a mutation in the PAR4 gene may include restriction fragment length polymorphism, hybridisation techniques, DNA sequencing, exonuclease reistance, microsequencing, solid phase extension using ddNTPs, extension in solution using ddNTPs, oligonucleotide assays, methods for detecting single nucleotide polymorphism such as dynamic allele-specific hybridisation. ligation chain reaction, mini-sequencing, DNA “chips”, allele-specific oligonucleotide hybridisation with single or dual-labelled probes merged with PCR or with molecular beacons, and others.

Analyzing the expression of the PAR4 gene may be assessed by any of a wide variety of well-known methods for detecting expression of a transcribed nucleic acid or translated protein.

In a preferred embodiment, the expression of the PAR4 gene is assessed by analyzing the expression of mRNA transcript or mRNA precursors, such as nascent RNA, of said gene.

Said analysis can be assessed by preparing mRNA/cDNA from cells in a biological sample from a subject, and hybridizing the mRNA/cDNA with a reference polynucleotide. The prepared mRNA/cDNA can be used in hybridization or amplification assays that include, but are not limited to, Southern or Northern analyses, polymerase chain reaction analyses, such as quantitative PCR (TaqMan), and probes arrays such as GeneChip™ DNA Arrays (AFF YMETRIX).

Advantageously, the analysis of the expression level of mRNA transcribed from the PAR4 gene involves the process of nucleic acid amplification, e.g., by RT-PCR (the experimental embodiment set forth in U.S. Pat. No. 4,683,202), ligase chain reaction (BARANY, Proc. Natl. Acad. Sci. USA, vol.88, p: 189-193, 1991), self sustained sequence replication (GUATELLI et al., Proc. Natl. Acad. Sci. USA, vol.57, p: 1874-1878, 1990), transcriptional amplification system (KWOH et al., 1989, Proc. Natl. Acad. Sci. USA, vol. 86, p: 1173-1177, 1989), Q-Beta Replicase (LIZARDI et al., Biol. Technology, vol. 6, p: 1197, 1988), rolling circle replication (U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

In another preferred embodiment, the expression of the PAR4 gene is assessed by analyzing the expression of the protein translated from said gene. Said analysis can be assessed using an antibody (e.g., a radio-labeled, chromophore-labeled, fluorophore-labeled, or enzyme-labeled antibody), an antibody derivative (e.g., an antibody conjugate with a substrate or with the protein or ligand of a protein of a protein/ligand pair (e.g., biotinstreptavidin)), or an antibody fragment (e.g., a single-chain antibody, an isolated antibody hypervariable domain, etc.) which binds specifically to the protein translated from the PAR4 gene.

Said analysis can be assessed by a variety of techniques well known from one of skill in the art including, but not limited to, enzyme immunoassay (EIA), radioimmunoassay (RIA), Western blot analysis and enzyme linked immunoabsorbant assay (RIA).

The method of the invention may comprise comparing the level of expression of the PAR2 gene in a biological sample from a subject with the normal expression level of said gene in a control. A significantly higher level of expression of said gene in the biological sample of a subject as compared to the normal expression level is an indication that the patient is predisposed to developing an influenza virus type A infection. The “normal” level of expression of the PAR2 gene is the level of expression of said gene in a biological sample of a subject not afflicted by any influenza virus type A infection. Preferably, said normal level of expression is assessed in a control sample (e.g., sample from a healthy subject, which is not afflicted by any influenza virus type A infection) and preferably, the average expression level of said gene in several control samples.

According to the present invention, the treatment or prevention of influenza virus type A infection in a subject is not the treatment or prevention of respiratory distress syndrome.

The invention will be further illustrated by the following figures and examples. However, these examples and figures should not be interpreted in any way as limiting the scope of the present invention.

### FIGURES

FIG. 1: Role of PAR4 in pathogenesis of IAV infection

A: Survival rate and weight evolution of mice infected with IAV treated/non-treated with PAR-4 agonist

B: Survival rate and weight evolution of mice uninfected with IAV and treated/non-treated with PAR4 agonist

C: Influence of administration of PAR4 agonist in the lung virus titers in mice infected by IAV.

FIG. 2: Role of PAR4 agonist in inflammation of the lungs of infected mice

A Evolution of BAL total protein in mice infected with IAV and treated/non-treated with PAR4 agonist

B: Evolution of BAL inflammatory cytokines in mice infected with IAV and treated/non-treated with PAR4 agonist

C: Histopathological examination of the lungs of mice infected with IAV and treated/non-treated with PAR4 agonist

FIG. 3: Role of PAR4 antagonist in inflammation and virus replication

A: Survival rate and weight evolution of mice infected with IAV and treated/non-treated with pepducin p4pal-10.

B: Evolution of BAL total protein and inflammatory cytokines in mice infected with IAV and treated/non-treated with pepducin p4pal-10.

### EXEMPLES

PAR4 Contributes to Pathogenesis of IAV Infection

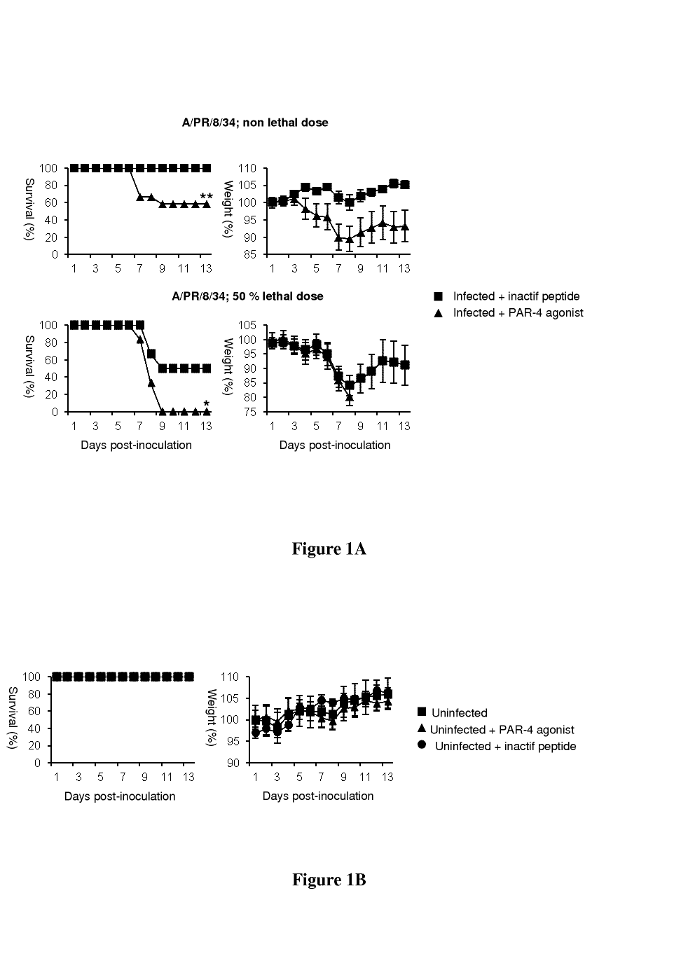

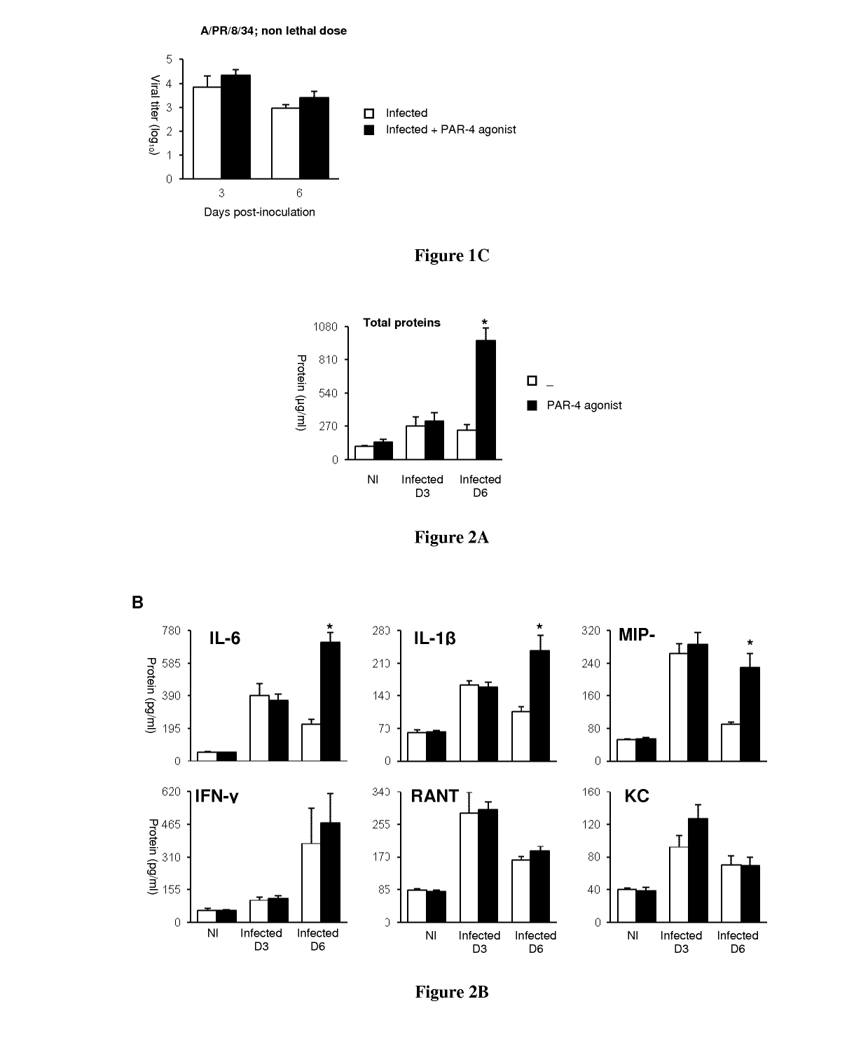

To investigate the role of PAR4 in pathogenesis of IAV infections, mice were inoculated with different PFU of IAV A/PR/8/34 (non lethal or 50% lethal doses) and either left untreated or stimulated with PAR4-activating peptides (100 μg). As shown in FIG. 1A, mice treated with PAR4-activating peptides significantly increased mortality rates and weight loss compared to untreated control mice. In contrast, treatment of uninfected mice with PAR4 agonists did not affect survival rates or body weight of mice, showing that the effect of PAR4 agonist on survival and weight loss required IAV infection (FIG. 1B). Thus, PAR4 activation led to increased pathogenicity of IAV infection.

PAR4 and Virus Replication

To gain further inside into the role of PAR4 in virus replication, mice were infected with IAV A/PR/8/34 (non lethal dose) and treated or not with PAR4 agonists (100 μg). Infectious virus titer was then evaluated in the lungs of infected mice. As shown in FIG. 1C, no significant differences in lung virus titers were observed 3 and 6 days post-inoculation between mice treated or not with PAR4 agonists. Thus, the deleterious effect of PAR4 agonists was most likely independent of virus replication in the lungs.

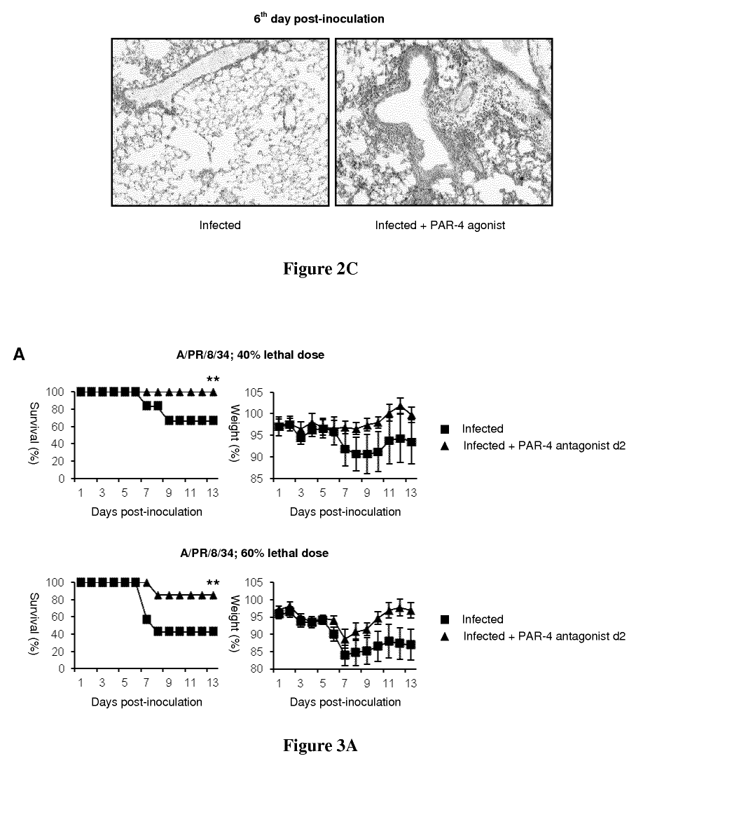

Agonists of PAR4 increase inflammation of the lungs of infected mice Because severe inflammation may account for IAV pathogenesis, we then investigated the role of PAR4 agonists in the inflammatory response induced by IAV infection. To this end, the amount of total protein was evaluated in the fluid of BAL (broncho-alveolar lavage) of infected mice (non lethal dose) treated or not with PAR4 agonists (100 μg). Compared to untreated mice, PAR4 agonist treatment significantly increase total proteins in the BAL at day 6 post-infection (FIG. 2A) as well as levels of IL-6, IL1-β, MIP-2 responses (FIG. 2B). This difference was not observed for IFN-γ, RANTES and KC cytokines. Also, histopathological examination showed that treatment with PAR4 agonist increased cellular infiltrates in the lungs from infected mice treated compared to untreated mice (FIG. 2C). Thus, PAR4 activation can increase IAV-induced production of specific cytokines in the lungs of infected mice.

PAR4 Antagonist Protects Against H1N1 Virus Infections

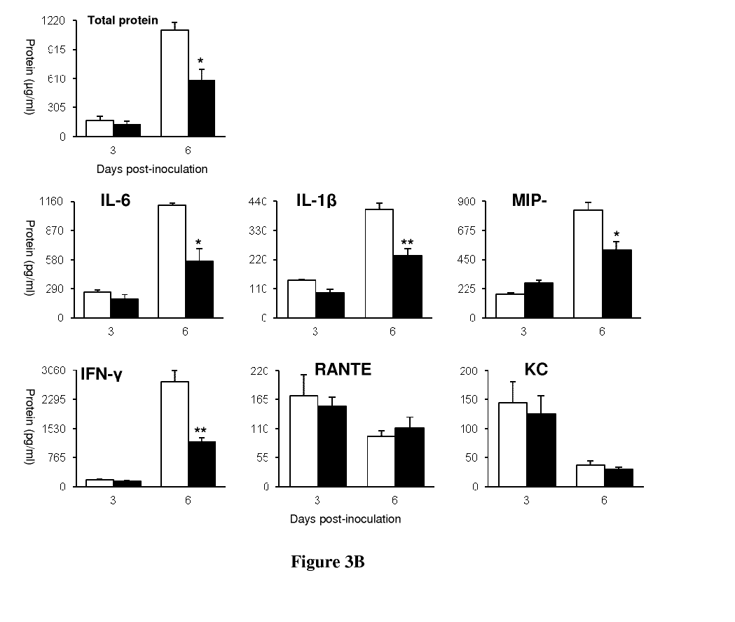

We next investigated whether pharmacological inhibition of PAR4-signaling might alter the course of IAV infection. To this end, we examined the effects of the PAR4 antagonist pepducin p4pal-10 on the course of IAV infection. When mice were infected with lethal doses of IAV A/PR/8/34 (H1N1) (40 or 60% lethal doses), treatment with pepducin p4pal-10 (10 μg) protected mice from weight loss and death (FIG. 3A). Thus, blocking PAR4 protected mice from IAV infection, consistent with the notion that PAR4 contributes to IAV pathogenesis in this model.

Inflammation and virus replication are attenuated by PAR4 antagonist We then determined whether blockade of PAR4 signaling would result in reduced IAV-induced inflammation in vivo. Mice were infected with lethal dose of IAV A/PR/8/34, treated or not with Pepducin p4pal-10, and BAL was collected. As shown in FIG. 3B, treatment with Pepducin p4pal-10 significantly reduced the level of total proteins as well as IL-6, IL1-β, MIP-2 and IFN-γ, in BAL at day 6 post-inoculation but not at day 3, as measured by ELISA. In contrast levels of RANTES and KC were similar in the BAL of infected mice whatever Pepducin p4pal-10 treatment.

1. A method of treating or preventing an influenza virus type A infection in a subject in need thereof, comprising
administering to said subject a therapeutically effective amount of a Protease-Activated Receptors-4 (PAR-4) antagonist.

2. The method according to claim 1, wherein said influenza virus type A is H1N1 virus.

3. The method according to claim 1, wherein said PAR-4 antagonist is selected from the group consisting of a peptide, a peptide mimetic, a small molecule organic compound, an aptamer, a pepducin, a polynucleotide or an antibody

4. The method according to claim 1, wherein said PAR-1 antagonist is a pepducin.

5. The method according to claim 4, wherein said pepducin is pepducin P4pal-10.

6. The method according to claim 1, wherein said subject is a mammal.

7. Pharmaceutical composition comprising
(i) at least one PAR-4 antagonist, and
(ii) at least one Protease-Activated Receptors-2 (PAR-2) agonist or at least one PAR-1 antagonist.

8. A method of treating or preventing an influenza virus type A infection in a subject in need thereof, comprising
administering to said subject a therapeutically effective amount of a pharmaceutical composition comprising
(i) at least one PAR-4 antagonist, and
(ii) at least one Protease-Activated Receptors-2 (PAR-2) agonist or at least one PAR-1 antagonist.

9. A method of treating or preventing an influenza virus type A infection in a subject in need thereof, comprising
administering to said subject a therapeutically effective amount of
(i) at least one PAR-4 antagonist, and
(ii) at least one Protease-Activated Receptors-2 (PAR-2) agonist or at least one PAR-1 antagonist.

10. The method of claim 6, wherein said mammal is a human.
